(19) United States
(12) Patent Application Publication
(10) Pub. No.: US 2011/0264178 A1
(43) Pub. Date: Oct. 27, 2011

(54) Probe for Neural Stimulation

(75) Inventors: Mehran Mehregany, San Diego, CA (US); Grant McCallum, South Euclid, OH (US); Noppasit Laotaveerungrueng, Cleveland, OH (US); Chia-Hua Lin, Shaker Heights, OH (US)

(73) Assignee: CASE WESTERN RESERVE UNIVERSITY, Cleveland, OH (US)

(21) Appl. No.: 13/095,340

(22) Filed: Apr. 27, 2011

Related U.S. Application Data
(60) Provisional application No. 61/328,498, filed on Apr. 27, 2010.

(51) Int. Cl. A61N 1/05

(52) U.S. Cl. 607/116

(57) ABSTRACT

A neural probe for stimulating neural tissue is disclosed. The probe comprises a three-dimensional arrangement of individually addressable electrodes. As a result, embodiments of the present invention can steer stimulative electric current through a wide range of paths through neighboring neural tissue. This enables specific targeting of neural selected neural tissue. In addition, embodiments of the present invention provide increased tolerance to probe misplacement or movement after insertion. Further, embodiments of the present invention enable changes in the neural tissue being stimulated without requiring additional surgical procedures.

### CROSS REFERENCE TO RELATED APPLICATIONS

This case claims priority of U.S. Provisional Patent Application U.S. 61/328,498, which was filed on Apr. 27, 2010 (Attorney Docket: 747-004US), and which is incorporated herein by reference.

### STATEMENT REGARDING FEDERALLY-SPONSORED RESEARCH

This invention was made with Government support under contracts NBCH1090004, 4714-10417, and 100112716, each of which was awarded by the United States Department of Defense—United States Army. The Government has certain rights in the invention.

### FIELD OF THE INVENTION

The present invention relates to medical devices in general, and, more particularly, to implantable neural probes.

### BACKGROUND OF THE INVENTION

Electrical stimulation of nerve tissue and recording of neural electrical activity form the basis of modern prostheses and treatment for a variety of neurological disorders, including spinal cord injury, stroke, sensory deficit, Parkinson's disease, and essential tremor control, among others. These procedures are affected by means of neural probes that are implanted directly into brain tissue so that electrodes located on the probes are in close proximity to nerve tissue. In some treatments, these electrodes are used to deliver patterns of electric pulses to stimulate the neural tissue. In other treatments, the electrodes are used receive electrical signals to monitor neural activity. Neural probes are also used in long-term implants, such as cochlear implants or other neural prostheses.

A typical conventional neural probe includes a flexible, circular lead having a linear arrangement of cylindrical electrodes located at its tip. A pulse generator connected to the lead delivers electrical pulses to each of the electrodes. Although such probes enable easy introduction into the neural tissue, they have several disadvantages.

First, since the electrodes are cylindrically shaped, they generate an electric field directed 360° around the lead. As a result, in many cases tissue in the region of the electrode is stimulated unintentionally. This can lead to many undesirable side effects, such as blurred speech, apathy, cognitive dysfunction, depression, incontinence, and sexual dysfunction.

Second, proper placement of a neural probe with respect to the targeted neural tissue is critical. Misalignment by only one millimeter (mm) can degrade therapy efficacy or induce deleterious side effects. A conventional neural probe is typically inserted through the skull of a patient and lodged in the neural tissue of the brain using CT scanning or MRI imaging to guide the surgeon. During surgery, however, the brain can shift slightly, enabling the probe to become displaced or dislodged.

Third, brain anatomy varies from patient to patient. As a result, the arrangement of the electrodes on the lead is often poorly matched to the functional structure of the neural tissue. Again, this can lead to unintended stimulation of non-targeted tissue and decreased efficacy of the stimulation of the targeted neural tissue.

A robust neural-stimulation probe that offers a high level of stimulation control would be a significant advance in the state-of-the-art.

### SUMMARY OF THE INVENTION

The present invention enables stimulation of specifically targeted neural tissue. Embodiments of the present invention comprise probes having a three-dimensional arrangement of electrodes, wherein the voltage on each electrode is independently controllable. As a result, probes in accordance with the present invention are able to perform current steering in directions that are unaligned with the longitudinal direction of the probe. This enables neural stimulation that is difficult, if not impossible, to achieve with prior-art neural stimulation probes.

An illustrative embodiment of the present invention is a probe that comprises four substrates that are arranged to form a column having a substantially square cross-section. Each substrate comprises a face that comprises a plurality of electrodes. The faces of the substrates are directed outwardly from a central longitudinal axis of the column. Since each electrode is independently addressable, the probe enables the flow of stimulative electrical current between any two electrodes. As a result, stimulative current flow can be directed along directions: aligned with a substrate face; aligned with the longitudinal direction; circumferentially about the longitudinal axis; or diagonally about the longitudinal axis.

In some embodiments, a probe comprises a sensor for sensing an environmental stimulus in the region of the probe, such as pressure, temperature, presence of a chemical, and the like.

In some embodiments, a probe comprises a recording electrode for sensing a neural signal.

In some embodiments, a probe comprises a processor for receiving signals from a recording electrode or sensor. In some embodiments, the processor includes additional circuitry, such as signal amplification circuitry, signal conditioning circuitry, and sensor control circuitry.

An embodiment of the present invention is a probe for stimulating neural tissue, the probe comprising: a first surface comprising a first plurality of electrodes, wherein the voltage on each of the first plurality of electrodes is independently controllable; and a second surface comprising a second plurality of electrodes, wherein the voltage on each of the second plurality of electrodes is independently controllable; wherein the first surface and second surface are arranged about a longitudinal axis through the body such that a first line extending normally outward from the first surface forms a non-zero angle with a second line extending normally outward from the second surface.

### BRIEF DESCRIPTION OF THE DRAWINGS

FIG. 1 depicts a schematic diagram of a cross-sectional view of a neural probe in accordance with the prior art.

FIGS. 2A and 2B depict schematic diagrams of a cross-sectional view and top view, respectively, of a neural probe in accordance with an illustrative embodiment of the present invention.

FIG. 3 depicts operations of a method suitable for fabricating a neural probe in accordance with the illustrative embodiment of the present invention.

FIGS. 4A-4C depict panel 202-1 at different stages of fabrication.

FIG. 5A depicts a schematic drawing of a top view of panel 202-1 in accordance with the illustrative embodiment of the present invention.

FIG. 5B depicts a schematic drawing of a top view of a panel in accordance with a first alternative embodiment of the present invention.

FIG. 5C depicts a schematic drawing of a top view of a tab in accordance with a second alternative embodiment of the present invention.

FIG. 6A depicts a schematic drawing of a top view of an end cap 204.

FIG. 6B depicts a photograph of a portion of a substrate comprising a plurality of fabricated end caps.

FIG. 7 depicts the steering of a flow of electric current by a neural probe in accordance with the illustrative embodiment.

### DETAILED DESCRIPTION

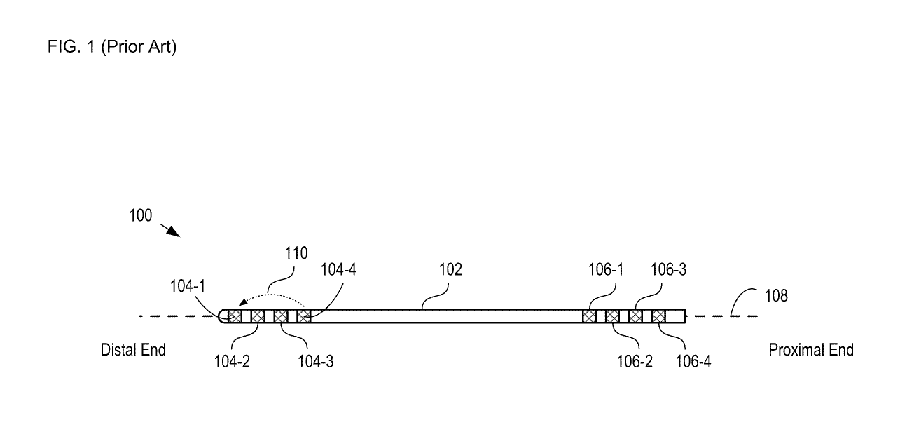

FIG. 1 depicts a schematic diagram of a cross-sectional view of a neural probe in accordance with the prior art. Probe 100 comprises lead 102, electrodes 104-1 through 104-4, and contacts 106-1 through 106-4.

Lead 102 is a flexible, hollow tube typically made of polyurethane and typically having a diameter of approximately 1.27 mm. Lead 102 encloses insulated interconnect wires (not shown for clarity) that electrically connect each of electrodes 104-1 through 104-4 (referred to, collectively, as electrodes 104) with a corresponding one of contacts 106-1 through 106-4 (referred to, collectively, as contacts 106).

Each of electrodes 104 is a cylinder of electrical conductor, typically comprising platinum and iridium, located at the distal end of lead 102. Electrodes 104 have a typical width of approximately 1.5 mm and are spaced apart by a space within the range of approximately 0.5 mm to approximately 1.5 mm. Typically spot-welding is used to connect electrodes 104 with their respective interconnect wires.

Each of contacts 106 is a cylinder of electrically conductive material that is electrical connected to one of electrodes 104 through one of the interconnect wires. Contacts 106 provide electrical connection points for an extension lead through which electrical signals can be provided to electrodes 104.

When energized, each electrode 104 generates an electric field that extends 360° about longitudinal axis 108. A voltage differential applied to two of electrodes 104 induces electrical current flow that flows along the length of lead 102. For example, a voltage differential between electrodes 104-1 and 104-4 induces an electric current along path 110. As a result, probe 100 provides only limited directional control over induced electrical current. Further, probe 100 stimulates any tissue within effective range of electrodes 104 or along a generated current flow. Probe 100, therefore, is not well suited to focused stimulation of specific neural tissue.

In contrast to prior art neural probes, embodiments of the present invention are capable of three-dimensional current steering by virtue of their inclusion of independently addressable electrodes on multiple non-co-planar surfaces. A probe in accordance with the present invention, therefore, can induce electric current flow that is directed along: 1) circumferential paths about the longitudinal axis of the probe; 2) paths aligned with the longitudinal axis of the probe; and 3) paths having directional components both aligned with and circumferential about the longitudinal direction of the probe.

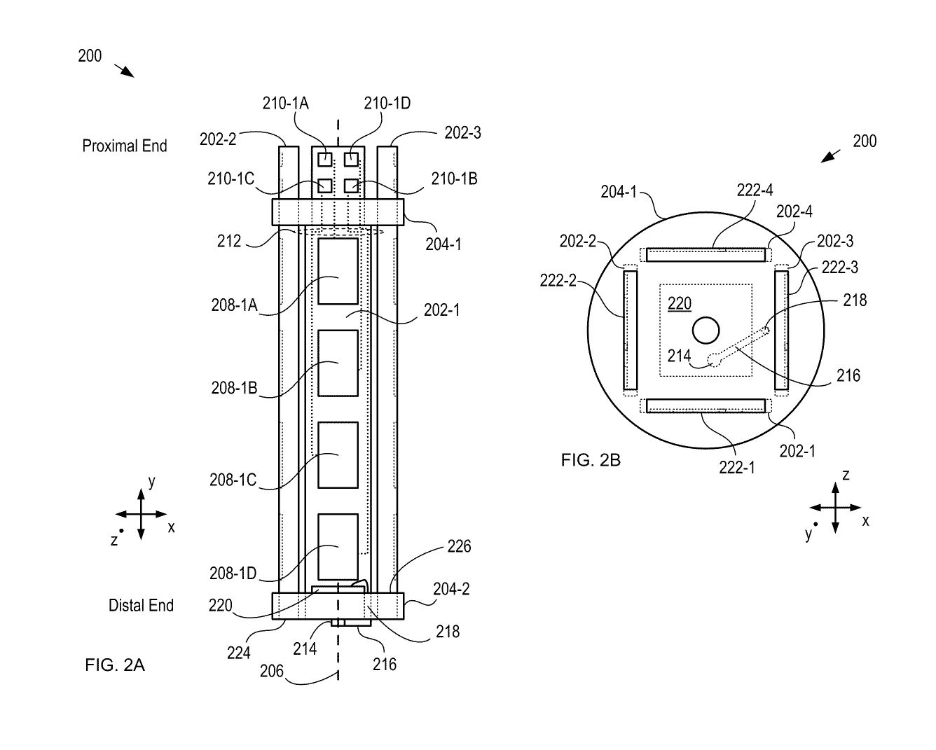

FIGS. 2A and 2B depict schematic diagrams of a cross-sectional view and top view, respectively, of a neural probe in accordance with an illustrative embodiment of the present invention. Probe 200 comprises panels 202-1 through 202-4, end caps 204-1 and 204-2, recording electrode 214, and processor 220.

Each of panels 202-1 through 202-4 (referred to, collectively, as panels 202) and end caps 204-1 and 204-2 (referred to, collectively, as end caps 204) are fabricated separately and then assembled to form probe 200. In some embodiments, micro-electro-mechanical systems (MEMS) fabrication technology is used to fabricate panels 202 and end caps 204.

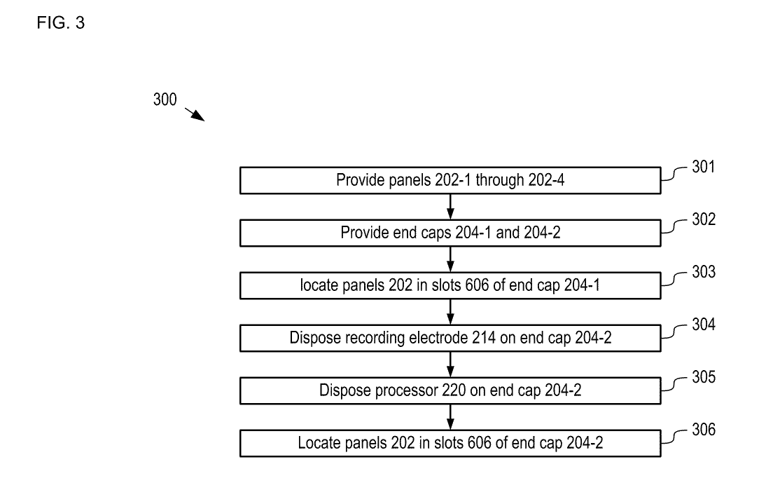

FIG. 3 depicts operations of a method suitable for fabricating a neural probe in accordance with the illustrative embodiment of the present invention. Method 300 begins with operation 301, wherein each of panels 202 is provided.

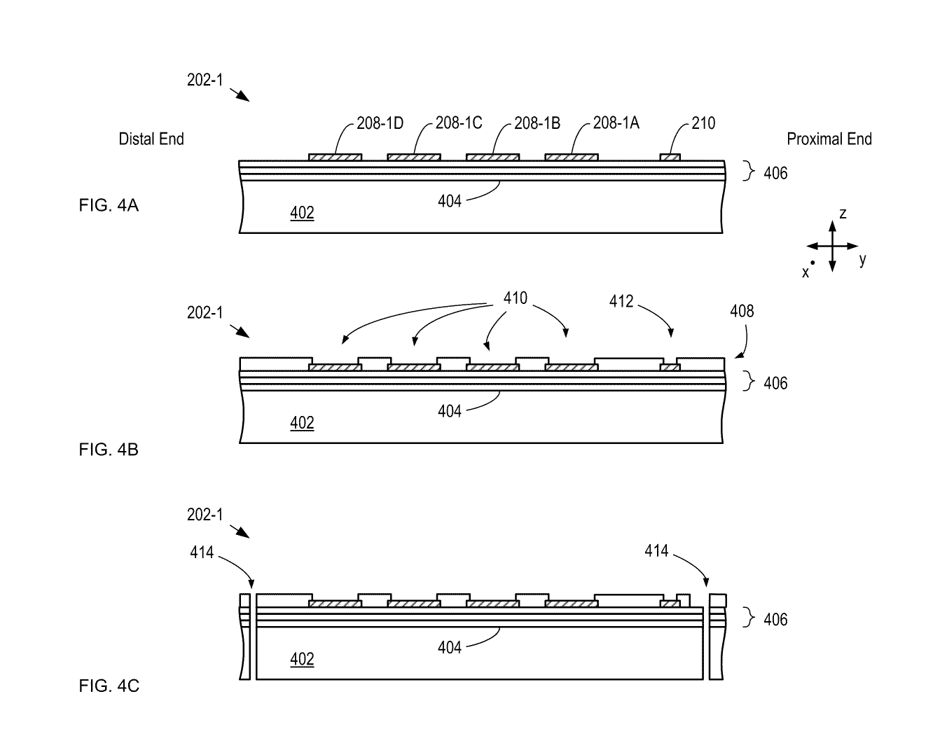

FIGS. 4A-4C depict panel 202-1 at different stages of fabrication. One skilled in the art will recognize that the fabrication of panel 202-1 is representative of the fabrication of each of panels 202.

Fabrication of panel 202 begins with deposition of a dielectric stack 406 on surface 404 of substrate 402.

Substrate 402 is a conventional p-doped, single-crystal silicon wafer that has a thickness of approximately 250 microns. In some embodiments, substrate 402 comprises another material suitable for use in a MEMS-based fabrication process. In some embodiments, substrate 420 has a different thickness within the range of 50 microns to 1000 microns.

Dielectric stack 406 comprises a tri-layer stack including low-temperature oxide (300 nm-thick) disposed on low-stress silicon nitride (150 nm-thick) disposed on low-temperature oxide (300 nm-thick), which is disposed on surface 404. The constituent layers of dielectric stack 406 are deposited using conventional low-pressure chemical vapor deposition (LPCVD). One skilled in the art will recognize that the number of layers, materials, and thicknesses for the constituent layers of dielectric stack 406 are matters of design choice and that alternative materials and thicknesses can be used without departing from the scope of the present invention.

Since silicon dioxide has a residual stress that is compressive and silicon nitride has a residual stress that is tensile, the alternating layers of dielectric stack 406 provide a degree of stress compensation that mitigates stress-induced bowing of substrate 402. Further, the stress compensation enables a greater composite thickness for dielectric stack 406, which improves electrical isolation of substrate 402 from electrically conductive elements disposed on dielectric stack 406.

After formation of dielectric stack 406, electrodes 208-1A through 208-1D (referred to, collectively, as electrodes 208), traces 212, and bond pads 210 are formed on dielectric stack 406 via conventional metal lift-off techniques. In some embodiments, electrodes 208, traces 212, and bond pads 210 are formed using conventional subtractive patterning of a deposited metal layer.

In the illustrative embodiment, each of electrodes 208 is a rectangle having dimensions of approximately 1500 microns by 500 microns. Electrodes 208 are arranged in a linear pattern and separated from one another by approximately 1300 microns. One skilled in the art will recognize that these dimensions and the arrangement of electrodes 208 are merely exemplary and that electrodes 208 can have any practical size, shape, and arrangement.

Each of electrodes 208, traces 212, and bond pads 210 comprises metals that are substantially biocompatible, such as platinum and titanium. In the illustrative embodiment, for example, each of electrodes 208, traces 212, and bond pads 210 comprises a layer of platinum having a thickness of approximately 300 nm, which is disposed on a layer of titanium having a thickness of approximately 10 nm. The titanium layer is formed between dielectric stack 406 and the platinum layer to enhance the adhesion of the metals to dielectric stack 406. One skilled in the art will recognize that this is only one possible combination of metals suitable for of electrodes 208, traces 212, and bond pads 210.

FIG. 4A depicts a schematic drawing of a cross-sectional view of panel 202-1 after the formation of electrodes 208, traces 212, and bond pads 210 on dielectric stack 206. Note that, for clarity, traces 212 are not depicted in FIGS. 4A-4C.

Probe 200 comprises a three-dimensional arrangement of individually addressable electrodes. This enables probe 200 to steer electric current in directions including directions aligned with longitudinal axis 206, circumferentially about longitudinal axis 206, and combinations thereof.

After the formation of electrodes 208, traces 212, and bond pads 210, passivation layer 408 is formed and patterned to open windows 410 over electrodes 208 and windows 412 over bond pads 210. Passivation layer 408 is a layer of silicon dioxide deposited via LPCVD and has a thickness of approximately 300 nm. Passivation layer 408 is patterned using conventional reactive-ion etching (RIE).

FIG. 4B depicts a schematic drawing of a cross-sectional view of panel 202-1 after the opening of windows 410 and 412 in passivation layer 408.

Panel 202-1 is separated from other components formed on the same wafer via the formation of trenches 414 in a two-stage RIE process. In order to form trenches, a first RIE is used to etch through dielectric stack 406 and a second deep-RIE is used to etch completely through substrate 402. In some embodiments, panel 202-1 remains connected to other components formed simultaneously via readily breakable retention tabs. In some embodiments, substrate 402 is etched in a two-stage deep-RIE process wherein the substrate is partially etched from the top (as shown in FIGS. 4A-C) and the turned over and etched from the backside to complete the release of panel 202-1.

FIG. 4C depicts a schematic drawing of a cross-sectional view of panel 202-1 after the formation of trenches 414.

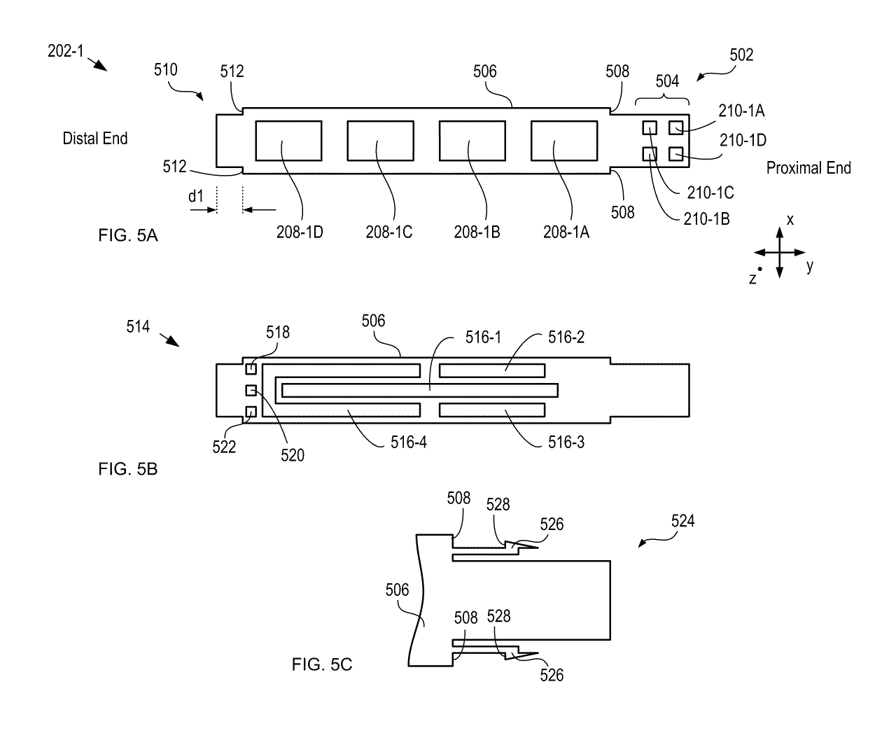

FIG. 5A depicts a schematic drawing of a top view of panel 202-1 in accordance with the illustrative embodiment of the present invention.

Panel 202-1 has a substantially rectangular shape that is approximately 824 microns wide by 11,500 microns long.

Panel 202-1 includes tabs 502 and 510, which determine the depths to which panel 202-1 engages with end caps 204-1 and 204-2, respectively.

Tab 502 has a width of approximately 724 microns and a length of approximately 1500 microns. Tab 502 comprises tab portion 504, on which bond pads 210-1A through 210-1D are disposed. Tab 502 locates the proximal end of panel 202-1 in end cap 204-1 such that tab portion 504 extends beyond the end cap, thereby providing access to the bond pads for attaching electrical leads during assembly of probe 200.

Tab 502 terminates at body 506 at shoulders 508. Each of shoulders 508 projects outward from tab 502 approximately 50 microns. Shoulders 508 collectively provide a mechanical stop that sets the insertion depth of panel 202-1 into end cap 204-1.

Tab 510 extends from body 508 by distance, dl, which is substantially equal to 250 microns (i.e., the thickness of end cap 204-2). Tab 510 terminates at body 506 at shoulders 512. Each of shoulders 512 projects outward from tab 510 approximately 50 microns. Shoulders 512 collectively provide a mechanical stop that sets the insertion depth of panel 202-1 into end cap 204-2.

FIG. 5B depicts a schematic drawing of a top view of a panel in accordance with a first alternative embodiment of the present invention. Panel 514 comprises electrodes 516-1 through 516-4, as well as sensors 518, 520, and 522. For clarity, panel 514 is depicted without bond pads and electrical traces. Each of electrodes 516-1 through 516-4 has a shape and position that is based on the characteristics of the neural tissue to be stimulated. It will be clear to one skilled in the art, after reading this Specification, that the size, position, and arrangement of electrodes 516-1 through 516-4 are merely exemplary and that myriad combinations of electrode sizes and configurations are possible.

Sensor 518 is a temperature sensor for monitoring the temperature at the insertion site of a neural probe in accordance with the present invention. It is desirable to monitor the temperature at the insertion site because tissue damage can occur due to inadvertent Joule heating caused by inducing a stimulation current that is too large.

Sensor 520 is a pressure sensor for monitoring insertion force during probe insertion into neural tissue. In addition sensor 520 enables monitoring of pressure at the insertion point after probe implant.

Sensor 522 is a chemical sensor, such as an ion-specific field-effect transistor, that provides an electrical signal in response to the presence of a target chemical or enzyme.

In some embodiments, panel 514 comprises monolithically integrated electronics, such as pre-amplification or signal conditioning circuitry, associated with one or more of sensors 218, 220, and 222. In some embodiments, a discrete electronics die is disposed on panel 514 and is electrically coupled with one or more of the sensors via conductive traces, through-wafer vias, or similar electrical interconnect.

FIG. 5C depicts a schematic drawing of a top view of a tab in accordance with a second alternative embodiment of the present invention. Tab 524 is analogous to tab 502; however, tab 524 comprises latches 526. Each of latches 526 is a portion of a panel that has been sculpted to define shoulder 528. Each of latches 526 is shaped such that it is resilient along the x direction. As a result, when a panel comprising latches 526 is inserted into an end cap, shoulders 508 and 528 collectively locate and capture the end cap to lock the panel and end cap together.

At operation 302, end caps 204-1 and 204-2 are provided.

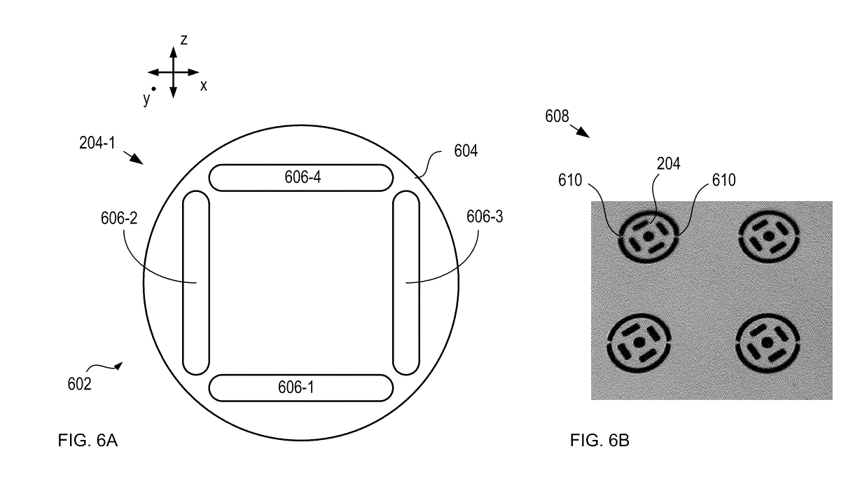

FIG. 6A depicts a schematic drawing of a top view of an end cap 204. End cap 204-1 comprises disc 604 and slots 606-1 through 606-4. End cap 204-1 is representative of end cap 204-2.

End caps 204 are fabricated by etching their pattern from substrate 602 using photolithography and deep-RIE. Substrate 602 is a conventional silicon wafer having a thickness of approximately 250 microns. In some embodiments, substrate 602 comprises a different biocompatible material and has a thickness other than 250 microns.

Disc 604 is a circular plate having a diameter sculpted from substrate 602 via conventional deep-RIE. In some embodiments, disc 602 has a different thickness within the range of approximately 50 microns to approximately 1000 microns.

Slots 606-1 through 606-4 (referred to, collectively, as slots 606) are through-channels having a width and length suitable for accepting one of tabs 502 or 510. As a result, in the illustrative embodiment, slots 606 are approximately 252 microns wide by 726 microns long. It should be noted that these dimensions are slightly larger than the dimensions of tabs 502 and 510 to facilitate insertion of the tabs into the slots.

FIG. 6B depicts a photograph of a portion of a substrate comprising a plurality of fabricated end caps. Substrate portion 608 includes four end caps 204 (shown including optional center holes), each of which is suitable for use as either an end cap 204-1 or end cap 204-2. Each of end caps 204 is held in place within substrate portion 608 via a pair of retention tabs 610. Retention tabs 610 are beams of substrate material that can be readily broken to remove end caps 204 from the substrate. It should be noted that retention tabs are typically formed during the fabrication of panels 202, as described above and with respect to FIGS. 4A-C.

At operation 303, panels 202 are located in slots 606 of end cap 204-1. Tabs 502 are inserted through each of slots 606 of end cap 204-1 until shoulders 508 abut end cap. While panels 202 and end cap 204-1 are held in their desired positions, adhesive is applied to affix them together.

At operation 304, recording electrode 214, trace 216, and through-wafer via 218 are added to end cap 204-2. Recording electrode 214 and trace 216 are disposed on a subset of end caps 204 during their fabrication from substrate 602, as discussed above. In order to electrically isolate electrode 214 and trace 216 from substrate 602, a layer of dielectric material, such as silicon dioxide, is first disposed on surface 224. Electrodes 214 and trace 216 are then formed on the subset of end caps 204 via conventional photolithography and metal lift-off technique. A through-wafer via 218 is also formed in each of the subset of end caps in conventional fashion.

At operation 305, processor 220 is disposed on surface 226 of end cap 204-2. Processor 220 is a conventional processor capable of receiving electrical signals from recording electrode 214. In some embodiments, processor 220 conditions the received electrical signals and amplifies them to facilitate their transmission to remotely located analysis equipment. Processor 220 is electrically connected to recording electrode 214 via through-wafer via 218 and a conventional bond, such as a wire bond, tab bond, and the like. In some embodiments, processor 220 includes a sensor for monitoring an environmental condition, such as temperature, pressure, the presence of a target chemical, etc. In some embodiments, processor 220 is formed on end cap 204-2 using conventional monolithic integration techniques.

At operation 306, panels 202 are located in slots 606 of end cap 204-2. Tabs 510 are inserted through each of slots 606 of end cap 204-2 until shoulders 512 abut end cap 204-2. While panels 202 and end cap 204-2 are held in their desired positions, adhesive is applied to affix them together.

It is an aspect of the present invention that planar microfabrication techniques can be used to fabricate planar components that are assembled to produce a three dimensional structure. It is a further aspect of the present invention that the planar components can be formed using either the same substrates and materials or different substrates and materials, without negatively impacting an ability to assemble them into a functional three-dimensional system.

One skilled in the art will recognize, after reading this Specification, that probe 200 is merely one example of a three-dimensional structure that can be formed in accordance with the present invention.

Once probe 202 is fully assembled, conductive leads are electrically connected to bond pads 210 to enable electrical communications between electrodes 202 and remotely located electronics, such electrical pulse generators, signal conditioners, and analytical equipment.

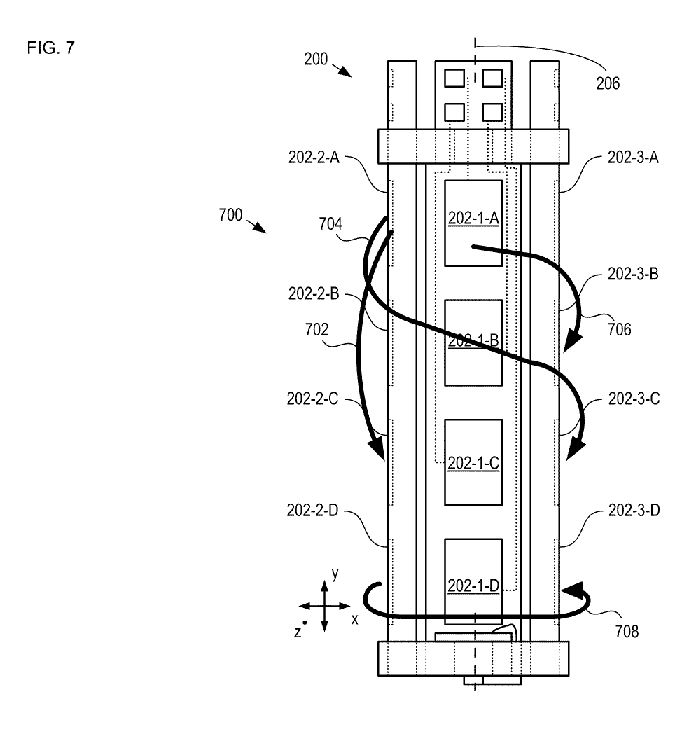

FIG. 7 depicts the steering of a flow of electric current by a neural probe in accordance with the illustrative embodiment. Current flow 700 is first induced by probe 200 by energizing electrodes 202-2-A and 202-2-C with a first voltage differential, which generates a first electric field between these electrodes. In response to the first electric field, current flow 700 arises through neural tissue in proximity to this electric field along current path 702. Current path 702 is directed along the y-direction (i.e., aligned with longitudinal axis 206). It should be noted that current flow along current path 702, therefore, is analogous to current flows that are attainable using neural probes known in the prior art.

By changing the electrodes across which the voltage differential is applied, current flow 700 is steered along a path other than current path 702. For example, by applying the voltage differential to electrodes 202-2-A and 202-3-C, current flow 700 changes its path and passes through different neural tissue in proximity to probe 200 along current path 704. Current path 704 follows a helical path that has a directional component along the y-direction (i.e., aligned with longitudinal axis 206) as well as a directional component that is circumferential about longitudinal axis 206. One skilled in the art will recognize that current path 704 represents a current flow path that is unattainable using neural probes known in the prior art, such as neural probe 100 described above and with respect to FIG. 1.

By changing the electrodes across which the voltage differential is applied once again (to electrodes 202-1-A and 202-3-B), current flow 700 is steered along current path 706, thereby passing through still different neural tissue in proximity to probe 200. Current path 706 also follows a helical path that has a directional component along the y-direction (i.e., aligned with longitudinal axis 206) as well as a directional component that is circumferential about longitudinal axis 206.

Still further, by changing the electrodes across which the voltage differential is applied to electrodes 202-2-D and 202-3-D), current flow 700 is steered along current path 708, which follows a circumferential path about longitudinal axis 206 as shown.

By virtue of an ability to steer electric current through a wide range of paths through neighboring neural tissue, embodiments of the present invention mitigate some of the disadvantages associated with neural probes of the prior art. Specifically, embodiments of the present invention provide increased tolerance to probe misplacement or movement after insertion. Further, embodiments of the present invention enable changes in the neural tissue being stimulated without requiring additional surgical procedures.

One skilled in the art will recognize, after reading this Specification, that current paths 702, 704, 706, and 708 represent only a few of the many current paths enabled by probes in accordance with the present invention.

It is to be understood that the disclosure teaches just one example of the illustrative embodiment and that many variations of the invention can easily be devised by those skilled in the art after reading this disclosure and that the scope of the present invention is to be determined by the following claims.

What is claimed is:

1. A probe for stimulating neural tissue, the probe comprising:
a first surface comprising a first plurality of electrodes, wherein the voltage on each of the first plurality of electrodes is independently controllable; and
a second surface comprising a second plurality of electrodes, wherein the voltage on each of the second plurality of electrodes is independently controllable;
wherein the first surface and second surface are arranged about a longitudinal axis through the body such that a first line extending normally outward from the first surface forms a non-zero angle with a second line extending normally outward from the second surface.

2. The probe of claim 1 further comprising:
a third surface comprising a third plurality of electrodes, wherein the voltage on each of the third plurality of electrodes is independently controllable; and
a fourth surface comprising a fourth plurality of electrodes, wherein the voltage on each of the fourth plurality of electrodes is independently controllable.

3. The probe of claim 1 further comprising:
a first panel having a first end and a second end, wherein the first panel comprises a first substrate that comprises the first surface; and
a second panel having a third end and a fourth end, wherein the second panel comprises a second substrate that comprises the second surface.

4. The probe of claim 3 further comprising a first end cap, the first end cap being dimensioned and arranged to locate the first end and the third end.

5. The probe of claim 4 further comprising a second end cap, the second end cap being dimensioned and arranged to locate the second end and the fourth end.

6. The probe of claim 1 wherein the first plurality of electrodes is arranged in a first arrangement on the first surface, and wherein the first arrangement is based on a characteristic of the neural tissue.

7. The probe of claim 1 wherein a first electrode of the first plurality of electrodes and a second electrode of the second plurality of electrodes are dimensioned and arranged to enable current flow in a first direction unaligned with the longitudinal axis.

8. The probe of claim 1 wherein a first electrode of the first plurality of electrodes and a second electrode of the second plurality of electrodes are dimensioned and arranged to enable current flow along a curved path about the longitudinal axis.

9. The probe of claim 1 further comprising a sensor.

10. The probe of claim 1 further comprising a recording electrode and a processor.

11. A probe for stimulating neural tissue, the probe comprising:
a first substrate, the first substrate comprising a first plurality of electrodes, wherein the voltage on each of the first plurality of electrodes is independently controllable;
a second substrate, the second substrate comprising a second plurality of electrodes, wherein the voltage on each of the second plurality of electrodes is independently controllable;
a third substrate, the third substrate comprising a third plurality of electrodes, wherein the voltage on each of the third plurality of electrodes is independently controllable; and
a fourth substrate, the fourth substrate comprising a fourth plurality of electrodes, wherein the voltage on each of the fourth plurality of electrodes is independently controllable;
wherein the first substrate, second substrate, third substrate, and fourth substrate are arranged about a first axis, and wherein the first substrate and the second substrate are not co-planar and are not parallel.

14. The probe of claim 11 wherein the first plurality of electrodes and the second plurality of electrodes enable a first current flow that is unaligned with the first axis.

15. The probe of claim 11 further comprising a sensor.

16. The probe of claim 11 further comprising a recording electrode.

17. The probe of claim 16 further comprising a processor, wherein the processor and the recording electrode are electrically coupled.

18. The probe of claim 11 further comprising a first end cap and a second end cap, wherein the first end cap and second end cap collectively align and locate each of the first substrate, second substrate, third substrate, and fourth substrate.

19. A probe for stimulating neural tissue, the probe comprising:
a first plurality of electrodes that are arranged in a first arrangement that is substantially linear along a first line, wherein the voltage on each of the first plurality of electrodes is independently controllable; and
a second plurality of electrodes that are arranged in a second arrangement that is substantially linear along a second line, wherein the voltage on each of the second plurality of electrodes is independently controllable;
wherein the first plurality of electrodes and second plurality of electrodes are collectively arranged about a first axis such that the first line, the second line, and the first axis are substantially parallel, and wherein the first plurality of electrodes and second plurality of electrodes collectively enable a first current flow that is in a first direction that is non-parallel with the first axis.

20. The probe of claim 19 further comprising a sensor.

21. The probe of claim 19 further comprising a recording electrode.

22. The probe of claim 21 further comprising a processor, wherein the processor and the recording electrode are electrically coupled.

23. The probe of claim 19 further comprising:
a third plurality of electrodes that are arranged in a third arrangement that is substantially linear along a third line, wherein the voltage on each of the third plurality of electrodes is independently controllable; and
a fourth plurality of electrodes that are arranged in a fourth arrangement that is substantially linear along a fourth line, wherein the voltage on each of the fourth plurality of electrodes is independently controllable;
wherein the third plurality of electrodes and fourth plurality of electrodes are collectively arranged about the first axis such that the third line, the fourth line, and the first axis are substantially parallel, and wherein the third plurality of electrodes and fourth plurality of electrodes collectively enable a second current flow that is in a second direction that is non-parallel with the first axis.

24. The probe of claim 19 wherein the first plurality of electrodes and second plurality of electrodes are arranged in a first arrangement that is based on a physical characteristic of the neural tissue.

25. The probe of claim 19 wherein at least one electrode of the first plurality of electrodes is characterized by a shape that is based on a physical characteristic of the neural tissue.
